(19) United States
(12) Patent Application Publication
(10) Pub. No.: US 2008/0078408 A1
(43) Pub. Date: Apr. 3, 2008

(54) URETHRAL CONDOMS AND LIQUID FEMALE CONDOMS

(76) Inventor: Benjamin Won Park, Kunsan (KR)

Correspondence Address: DAVIS WRIGHT TREMAINE LLP/Los Angeles, 865 FIGUEROA STREET, SUITE 2400, LOS ANGELES, CA, 90017-2566, US

(21) Appl. No.: 11/874,030

(22) Filed: Oct. 17, 2007

Related U.S. Application Data
(63) Continuation-in-part of application No. PCT/KR2006/001361, filed on Apr. 13, 2006.

(30) Foreign Application Priority Data
Apr. 18, 2005 (KR) 10-2005-0031818
Jun. 20, 2005 (KR) 10-2005-0052809
Jul. 14, 2005 (KR) 10-2005-0063697
Aug. 3, 2005 (KR) 10-2005-0070960
Apr. 10, 2006 (KR) 10-2006-0032134
Apr. 11, 2006 (KR) 10-2006-0032586

(51) Int. Cl. A61F 6/06; A61F 6/02; A61K 33/38; A61M 31/00; A61P 15/00

(52) U.S. Cl. 128/832; 128/830; 128/842; 424/618; 604/515; 604/516

(57) ABSTRACT

Lubricating gel compositions manufactured from water-soluble polymers and distilled water can prevent not only pregnancy but also sexually transmitted diseases (STDs) including AIDS due to their roles as physical blocking walls and spermicides. Colloidal silver, which has germicidal effects in a wide range, can be added to these compositions. These lubricating gel compositions, which are harmless and non-irritating to the male urethra, can be called “Urethral Condoms” because they act like condoms when injected into male urethras, and are easily eliminated from urethras by urination. They can also be called “Liquid Female Condoms” because they act like female condoms when injected into vaginas, and are easily eliminated from vaginas by vaginal irrigators and the vaginal scrubbing sponge of this invention.

This application is a Continuation-in-Part of International Application PCT/KR2006/001361, filed Apr. 13, 2006, which designated the U.S. and that International Application was published under PCT Article 21(2) in English, which claims a benefit of priority under 35 U.S.C. §119(a) from Korean Patent Application No. 10-2005-0031818, filed Apr. 18, 2005, Korean Patent Application No. 10-2005-0052809, filed Jun. 20, 2005, Korean Patent Application No. 10-2005-0063697, filed Jul. 14, 2005, Korean Patent Application No. 10-2005-0070960, filed Aug. 3, 2005, Korean Patent Application No. 10-2006-0032134, filed Apr. 10, 2006, and Korean Patent Application No. 10-2006-0032586, filed Apr. 11, 2006.

### FIELD OF INVENTION

The present invention relates to compositions and devices which can be used during sexual intercourses, wherein these compositions act not only as spermicides and germicides but also as physical blocking walls for sperms and germs. Therefore, these compositions can be used to prevent both pregnancy and sexually transmitted diseases (STDs) including acquired immunodeficiency syndrome (AIDS).

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Presently, nonoxynol-9 is the most widely used spermicide for contraception. However, it is known to cause inflammations and ulcers in the vagina when used too often. Although many other spermicides such as octoxynol-9, menfegol, and suramin (see e.g., U.S. Patent Publication No. 20050143461) have been found, there is no spermicide that is completely harmless to the human body. This is because a spermicide toxic enough to kill sperms is toxic to some extent to the human body as well.

Presently, the best way to prevent various STDs and AIDS is putting on a condom or a female condom, which acts as a physical blocking wall between germs and human epithelia. However, condoms are generally detested due to diminution in the sexual feeling. On the contrary, lubricating gel compositions increase the sexual feeling. However, ordinary lubricating gel compositions alone are of little help in preventing STDs and AIDS. Therefore, various lubricating gel compositions containing chemical germicides have been developed for the purpose of preventing STDs and AIDS. For example, lubricating gel compositions containing germicides such as silver sulfadiazine (see e.g., U.S. Pat. No. 4,952,411), chlorhexidine salt (see e.g., U.S. Pat. No. 6,624,198), zinc salts (see e.g., U.S. Pat. No. 5,980,477), sodium dodecyl sulfate (see e.g., U.S. Pat. No. 6,635,242) and dextrin sulfate (see e.g., U.S. Pat. No. 6,821,958) have been developed. Colloidal silver is also known to be an effective germicide that can eradicate within several minutes more than 650 disease-causing bacteria and viruses. However, it has not been tried yet contained in lubricating gel compositions for prevention of STDs and AIDS.

Bergeron et al. (U.S. Pat. No. 6,068,851) disclosed a new concept of semi-solid female condom as follows: thermo-reversible liquids such as poloxamers applied on vaginal surfaces solidify and act as a protective semi-solid layer against pathogens. However, it was not tested whether this semi-solid layer was strong enough to withstand vigorous frictional movements of sexual intercourse and whether this semi-solid layer interferes with sexual feelings, such as a condom would. Furthermore, those thermo-reversible liquids by themselves have not been proved for any contraceptive functions.

Presently, a catheter made of a long and thin rubber tube has to be inserted deep into the male urethra to inject liquid chemical agents into the male urethra, the prostate gland and the urinary bladder in order to diagnose or treat diseases in them. However, most people detest this operation due to a severe pain caused by insertion of a solid tube into the extremely sensitive urethra. Thus far, no device or method is known to inject liquid deeply into the male urethra without inserting a solid tube into it.

Presently, the most widely used vaginal irrigator is composed of an elastic bottle and a spray nozzle which form a straight line. It is uncomfortable to use it because one should be in the standing or squatting position. It is inconvenient to use it because air often comes out of the elastic bottle instead of the washing liquid because washing liquid is sprayed upward while the spray nozzle is inserted into the vagina. It is also messy to use it because hands can get wet easily with the spilled washing liquid.

In order to cleanse the vagina with water using the above-mentioned vaginal irrigator, a vaginal scrubber is needed for efficient cleansing like rubbing and scrubbing the skin is needed when a person takes a shower. In vaginal douches, most females use one of their fingers to scrub vaginal surfaces, which is unhygienic and injuries can be caused by the long nails. So far three different types of vaginal scrubbing sponges, which do not accompany injection of washing liquid, have been proposed. Basically, they were all composed of a piece of sponge and a handle to hold it. However, the ways to connect the sponge to the handle were all different. In U.S. Pat. No. 1,256,831 by Rogers, the sponge was bound with non-elastic thread or wire; in U.S. Pat. No. 3,228,398 by Leonard et a/, the sponge was bonded with adhesives; and in U.S. Pat. No. 7,112,184B2 by Bichsel et al., the sponge was held between two circular disks. The first and the third method do not give a smooth connecting surface between the sponge and the handle. In the second method, the hardened rough adhesive can be exposed and can touch the vaginal surface during the scrubbing movement, which would cause scratches or displeasure on vaginal surfaces.

In view of the present technical status and problems as discussed above, it can be said that there is a need in the art for the development of compositions that can act as both spermicides and germicides, that can also form physical blocking walls against both sperms and germs on human epithelia like condoms, and that are harmless and non-irritating to the vagina and the male urethra. There is also a need in the art for the development of a liquid injector that can be used without causing pains to inject the compositions into the male urethra, which is the most or solely susceptible to microbial invasion for a male. Further, there is also a need in the art for the development of a vaginal irrigator that can be used to wash out the injected compositions from the vagina while sitting comfortably on the toilet seat, which does not spray air instead of water, and which does not spill water onto hands. Additionally, there exists a need for the development of a vaginal scrubber that can be used to scrub off the injected compositions from the vagina and that does not cause scratches or displeasure on vaginal surfaces.

### SUMMARY OF THE INVENTION

The following embodiments and aspects thereof are described and illustrated in conjunction with compositions and methods which are meant to be exemplary and illustrative, not limiting in scope.

The present invention provides a composition useful for preventing pregnancy and/or preventing the contraction of sexually transmitted diseases (STDs), comprising: a quantity of distilled water; and a water-soluble polymer, wherein the concentration of the water-soluble polymer is about 0.5% to about 20.0% by weight. The water-soluble polymer may be polyvinyl alcohol (PVA) or a cellulose derivative such as sodium carboxymethyl cellulose (SCMC), hydroxyethyl cellulose (HEC), hydroxypropyl methyl cellulose (HPMC) or combinations thereof. The composition may further comprise a quantity of colloidal silver to bring the concentration of the colloidal silver to about 1 to about 10 ppm by weight.

The present invention also provides a method of using the composition to prevent or reduce the likelihood of pregnancy and/or to prevent or reduce the likelihood of contracting STDs, comprising: providing a composition of the present invention and applying the composition onto or into to a male urethra or into a vagina prior to sexual intercourse.

The present invention also provides a device for injecting a composition into the male urethra, comprising: an injection nozzle adapted for insertion into a male urethra; and a pressure generator, wherein the diameter of the distal end of the injection nozzle is smaller than the diameter of the male urethral outlet, the diameter of the proximal end of the injection nozzle is larger than the diameter of the male urethral outlet and the diameter of the injection nozzle increases from the distal end to the proximal end whereby a gap between the urethral outlet and the injection nozzle is sealed when the distal end of the injection nozzle is partially inserted into the urethra and pressed against the urethral outlet. The shape of the injection nozzle may be substantially similar to the shape of one-half of an ellipsoid. The ellipsoid may be defined by a first radius that is about 4 to about 9 mm, a second radius that is about 5 to about 12 mm and a third radius that is about 10 to about 30 mm.

The present invention also provides a method of using the device for injecting the composition into the male urethra to prevent or reduce the likelihood of contracting a sexually transmitted disease, comprising: providing the device for injecting a composition into the male urethra and a composition comprising of the present invention; placing the composition into device; and using the device to inject the composition into the male urethra.

The present invention also provides a vaginal irrigation device to remove the composition from the vagina, comprising: a spray nozzle; a neck of the spray nozzle; and optionally, an elastic bottle, wherein the neck of the spray nozzle is removably connected to the spray nozzle and optionally, removably connected to the elastic bottle, and wherein the shape of the neck of the spray nozzle comprises a straight segment of about 10 mm to about 60 mm measured from the distal end, followed by a sharp curve of about 60 to about 120 degrees, followed by a first gradual curve of about 10 to about 90 degrees in the direction of the distal end, followed by a second gradual curve of about 0 to about 60 degrees in the opposite direction. The length of the spray nozzle may be about 10 to about 50 mm and the length of the neck of the spray nozzle is about 100 to about 400 mm. The neck of the spray nozzle may further be connected to a water pipe with a flexible hose which may comprise a pressure control valve. The neck of the spray nozzle may be made of a strong and pliable material.

The present invention also provides a method of using the vaginal irrigation device to remove the composition from the vagina, comprising: providing the vaginal irrigation device and using the vaginal irrigation device to cleanse the vagina. The method may further comprise using the vaginal irrigation device to remove the composition comprising distilled water and a water-soluble polymer from the vagina.

The present invention also provides a vaginal scrubbing sponge device to remove the composition from the vagina, comprising: a sponge; a handle; and a wide elastic ring or a single-sided adhesive tape, wherein the sponge is connected to the distal end of the handle with the wide elastic ring or the single-sided adhesive tape whereby the sponge is partially compressed on the proximal edge. The diameter of the portion of the handle wound with the wide elastic ring or the single-sided adhesive tape may be smaller than the diameter of an adjacent portion of the handle so that the thickness becomes even when the ring or the tape is wound. The vaginal scrubbing sponge device may further comprise a surface-coating material capable of filling a gap formed between the edge of the wide elastic ring or the single-sided adhesive tape and the handle to make a smooth connection between the sponge and the handle. The shape of the distal end of the handle may be spherical or substantially spherical, or wherein the diameter of the distal end of the handle is larger than the diameter of a portion of the handle that is adjacent to the distal end.

The present invention also provides a method of using the vaginal scrubbing sponge device to remove the composition from the vagina, comprising: providing the vaginal scrubbing sponge device and using the vaginal scrubbing sponge device to cleanse the vagina. The method may further comprise using the vaginal scrubbing sponge device to remove the composition comprising distilled water and a water-soluble polymer from the vagina.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 shows schematically the effects of physical blocking walls formed by adsorbed water-soluble polymer molecules on germs and human epithelia (polymer molecules are packed more densely than depicted because the intervals between polymer chains are 4-15 nm, which are a lot narrower than the size of HIV (diameter=125 nm), Neisseria Gonorrhoeae (diameter=1,100 nm), and sperms (diameter of head=2,200-6,000 nm, total length=50,000 nm)) in accordance with various embodiments of the present invention. Explanation of Signs: 1. Neisseria Gonorrhoeae, 2. HIV, 3. Human Epithelium, 4. Water-soluble Polymer Molecules Adsorbed on Neisseria Gonorrhoeae, 5. Water-soluble Polymer Molecules Adsorbed on HIV, 6. Water-soluble Polymer Molecules Adsorbed on Human Epithelium, 7. Pili of Neisseria Gonorrhoeae, 8. Adsorption Points of Water-soluble Polymer Molecules.

FIG. 2A shows the display of fifteen vaginal segments of swine in accordance with various embodiments of the present invention.

FIG. 2B shows the vaginal segments of FIG. 2A on which water, corn syrup, PVA-gel, SCMC-gel and HEC-gel were applied in each column in accordance with various embodiments of the present invention.

FIG. 2C shows the vaginal segments of FIG. 2B on which the blue pigment, the violet pigment and the blue dye ink were applied on the first, second and the third row respectively, in accordance with various embodiments of the present invention. They were rubbed 50 times vigorously with an index finger being pressed simulating the frictional movement during coitus.

FIG. 2D shows the vaginal segments of FIG. 2C after being rinsed with running water in accordance with various embodiments of the present invention.

FIG. 2E shows the vaginal segments of FIG. 2D after being scrubbed and cleaned with the vaginal scrubbing sponge in accordance with various embodiments of the present invention. Explanation of Signs: PIG-1=Blue pigment, PIG-2=Violet pigment, DYE=Blue dye ink, WATER=Water, SYRUP=Corn syrup, PVA-gel=Composition made from PVA, SCMC-gel=Composition made from SCMC, HEC-gel=Composition made from HEC.

FIG. 3A shows the cross-sectional view of the injection nozzle of the liquid injector in accordance with various embodiments of the present invention. Explanation of Signs: 1. Injection Nozzle, 2. Outlet of the Injection Nozzle, 3. Connecting Hole to the Syringe, 4. Half of the Ellipsoid.

FIG. 3B shows the liquid which is injected deep into the male urethra with the liquid injector in accordance with various embodiments of the present invention. Explanation of Signs: 1. Injection Nozzle, 6. Glans Penis, 7. Collapsed Urethra, 9. Expanded Urethra, 10. Injected Liquid into Urethra, 11. Syringe.

FIG. 4A shows the cross-sectional view of the female body with the spray nozzle inserted into the vagina in accordance with various embodiments of the present invention.

FIG. 4B shows the cross-sectional view of the female body when the anus is to be cleansed in accordance with various embodiments of the present invention. Explanation of Signs: 1. Elastic Bottle, 2. Neck of Spray Nozzle in the Shape of the Cobra, 3. Spray Nozzle, 5. Anus, 6. Colon, 8. Uterus, 9. Urinary Bladder, 10. Abdomen, 11. Toilet Seat, 12. Toilet.

FIG. 5 shows a way how the vaginal irrigator is connected to water pipes in accordance with various embodiments of the present invention. Explanation of Signs: 2. Neck of Spray Nozzle, 3. Spray Nozzle, 4. Sprayed Water, 31. Hot Water, 32. Cold Water, 33. Water Mixing Valve, 34. To Bath Tub, 35. To Shower Head, 36. Flexible Hose, 37. Pressure Control Valve.

FIG. 6 shows the cross-sectional diagram of the vaginal scrubbing sponge in accordance with various embodiments of the present invention. Explanation of Signs: 1. Sponge made of soft, elastic, absorptive and adsorptive material, 2. Handle, 3. Adhesive tape or wide elastic ring, 4. Edge of adhesive tape or wide elastic ring, 5. End of handle in the shape of sphere or the likes.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs.

The compositions of the present invention, which are completely harmless and non-irritating, can contribute greatly to the safety and freedom of the human sexual activities. The technical needs as mentioned in the Background of this invention are mostly satisfied by the present inventor as described herein. This invention can be immediately utilized industrially because all raw materials necessary for this invention are presently mass-produced.

Compositions for Prevention of Pregnancy and STDs

The present inventor discovered a surprising fact for the first time that distilled water is an excellent spermicide. This fact was proved experimentally by the present inventor by observing semen with an optical microscope after mixing with distilled water as described in the examples as Experiments 1-5, while it can be explained theoretically with the osmotic shock.

Furthermore, the present inventor discovered another surprising fact for the first time that water-soluble polymer molecules form impenetrable physical blocking walls for sperms and germs on human epithelia. This fact was proved experimentally as described in the examples as EXPERIMENT 6, while it can be explained theoretically as below.

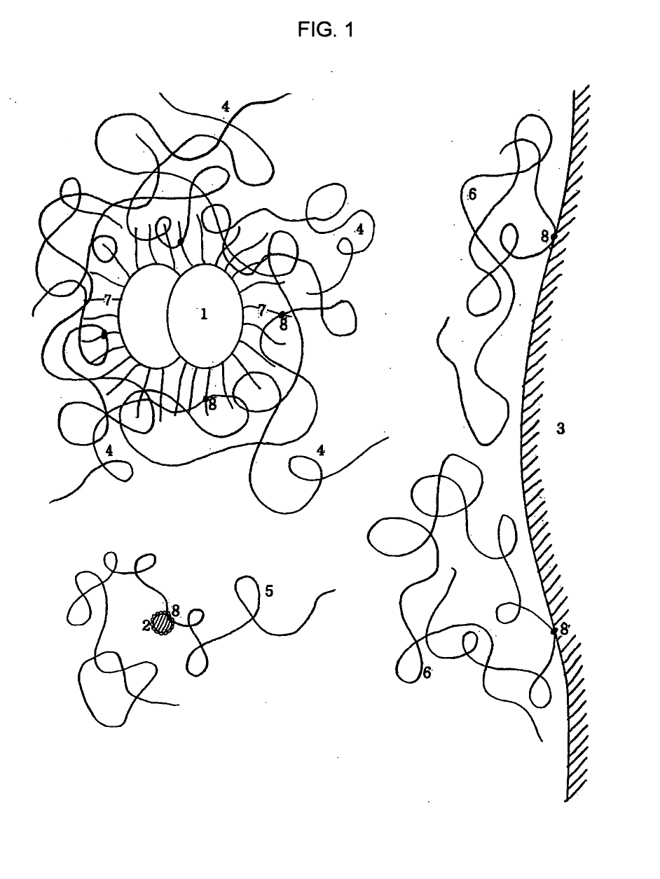

Water-soluble polymer molecules are known to form three-dimensional networks in water solution. Examples of water-soluble polymers include but are not limited to polyvinyl alcohol (PVA) and cellulose derivatives such as sodium carboxymethyl cellulose (SCMC), hydroxyethyl cellulose (HEC), and hydroxypropyl methyl cellulose (HPMC). The average interval between these polymer chains in water is estimated to be 4-15 nm (e.g. 13.8 nm for 2.9 wt % SCMC solution, 4.1 nm for 11.6 wt % PVA solution), which is a lot narrower than the size of sperms (diameter of head=2,200-6,000 nm, total length=50,000 nm), Neisseria Gonorrhoeae (diameter=1,100 nm) and HIV (human immunodeficiency virus, diameter=125 nm). Furthermore, water-soluble polymer molecules are ionic or polar so that they adsorb preferentially on human epithelia, sperms and microbes which carry ions or polarity on their surface protein molecules as shown in FIG. 1. Therefore, water-soluble polymer molecules are more densely packed on these surfaces than in the space. This is how the so-called physical blocking walls are formed on human epithelia. Not to mention sperms, almost all disease-causing microbes cannot easily penetrate through these densely packed physical blocking walls in order to adhere to human epithelia. This situation is similar to a ball on a lawny ground where the ball cannot touch the ground due to the thick and dense lawn. On the other hand, water-soluble polymer molecules adsorbed on sperms and germs would not only make them trapped in the space but also make their effective sizes a lot larger than their actual sizes as shown in FIG. 1 so that their penetration through the physical blocking walls becomes even more difficult. These unique physical properties against sperms and germs are possible only for water-soluble polymers.

Therefore, compositions manufactured from water-soluble polymers and distilled water not only have a spermicidal action but also form physical blocking walls against sperms and germs on human epithelia. They are different from the semi-solid female condom proposed by Bergeron et al. (U.S. Pat. No. 6,068,851) as follows: (1) they are injected as ready-made gels, (2) they are not broken or taken away because they are free-flowing and strongly adsorbed liquids, (3) they increase sexual feelings rather than decrease because they are excellent lubricants per se, (4) they are spermicides per se, and (5) they are composed of very safe materials enough to be edible and can be injected into the male urethra, which is extremely sensitive.

In order to further increase their prophylactic effects against STDs, colloidal silver which has a wide range of germicidal action can be added. Distilled water containing colloidal silver of 2-15 nm sizes and 1-10 ppm concentrations can be easily produced by electrolysis with a silver electrode of more than 99.99% in purity. It was discovered by the present inventor that colloidal silver does not cause any irritation to the male urethra.

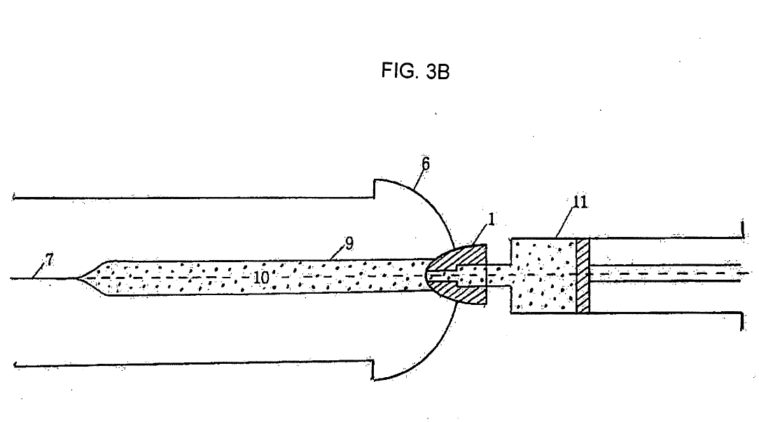

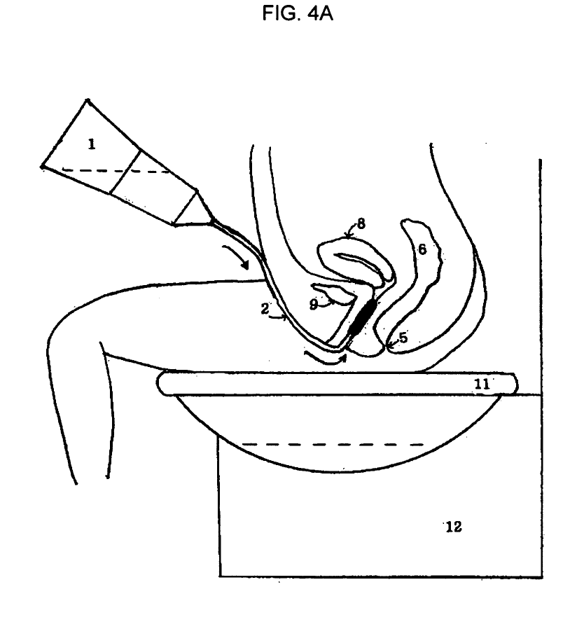

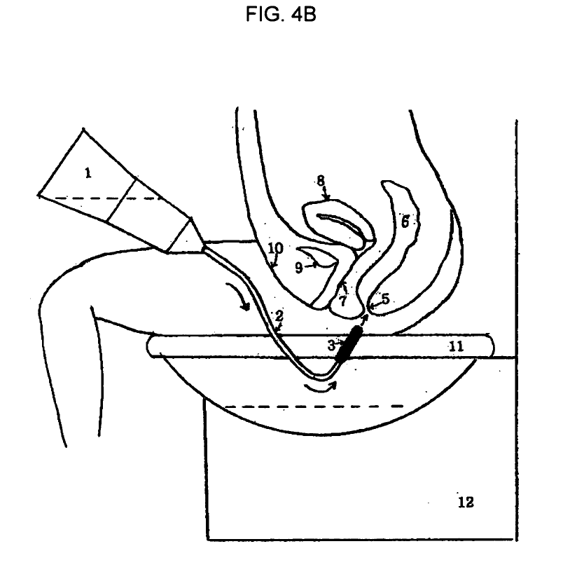

In practice, the compositions of the present invention, which are non-irritating to the male urethra, can be used a lot more efficiently by injecting them into the male urethra in order to protect the urethral epithelia, which are the most or solely susceptible to microbial attacks. This idea was proposed for the first time by the present inventor. This can be easily accomplished with the liquid injector of the present invention as shown in FIG. 3B. Therefore, these liquid products can be called “Urethral Condoms” because they act like condoms in the male urethra. Once injected into the male urethra, the compositions ooze out slowly toward the urethral outlet by the internal pressure in the urethra so that the urethral outlet is always filled with the compositions. Further, any viscous, nontoxic and non-irritant liquids can be used as urethral condoms as far as protection of the male urethra is concerned because no pathogen can travel against the bulk flow of viscous liquids through the male urethra where there is no mixing or turbulence even during sexual intercourse. Similarly, the compositions injected into vaginas can be called “Liquid Female Condoms”. Urethral Condoms can be easily washed out by urination while Liquid Female Condoms can be washed out with the vaginal irrigator and the vaginal scrubbing sponge of the present invention as shown in FIGS. 4A, 5 and 6. Liquid Female Condoms need not be washed out in a hurry from the vagina because the sperms and germs trapped in the three-dimensional network of water-soluble polymer molecules as shown in FIG. 1 will stay trapped without doing anything until they get washed out.

Compared with ordinary condoms, Urethral Condoms and Liquid Female Condoms have many advantages as follows: (1) they raise sexual feelings rather than reduce, (2) they cause no known allergies, (3) they cannot have structural defects such as pin holes, (4) they do not break, (5) they will not be mistakenly taken off, (6) a female can inject the compositions several hours before coitus, (7) they are not easily noticeable, (8) they can be used to protect much wider areas than condoms or female condoms because they can be easily applied in, on, and around both genitals and anuses.

Liquid Injector into the Male Urethra

As shown FIGS. 3A and 3B, the injection nozzle (1) has the shape of or similar to one half of the ellipsoid (e.g., the shape of a Rugby ball) whose radius are about 4-9 mm, 5-12 mm, and 10-30 mm respectively, which are all larger than the radius of the urethral outlet. The injection nozzle is made thinner on one side and thicker on the other side than the urethral outlet. Therefore, when the thinner end of the nozzle is inserted into the male urethra, it enters only partly into the male urethra. When the partly inserted nozzle is pressed slightly against the urethral outlet, the gap between the urethral outlet and the nozzle can be completely sealed. Then, the liquid is injected slowly into the male urethra by pressing the piston of the syringe. Then, the urethra turns from the collapsed state (7) to the expanded state (9). As the injection continues the injected liquid (10) can reach to the prostate gland and the urinary bladder. Instead of a syringe, a collapsible tube similar to a toothpaste tube can be used as a pressure generator. With minor modification, this liquid injector can be used to inject the compositions of this invention into the vagina as well.

Vaginal Irrigators

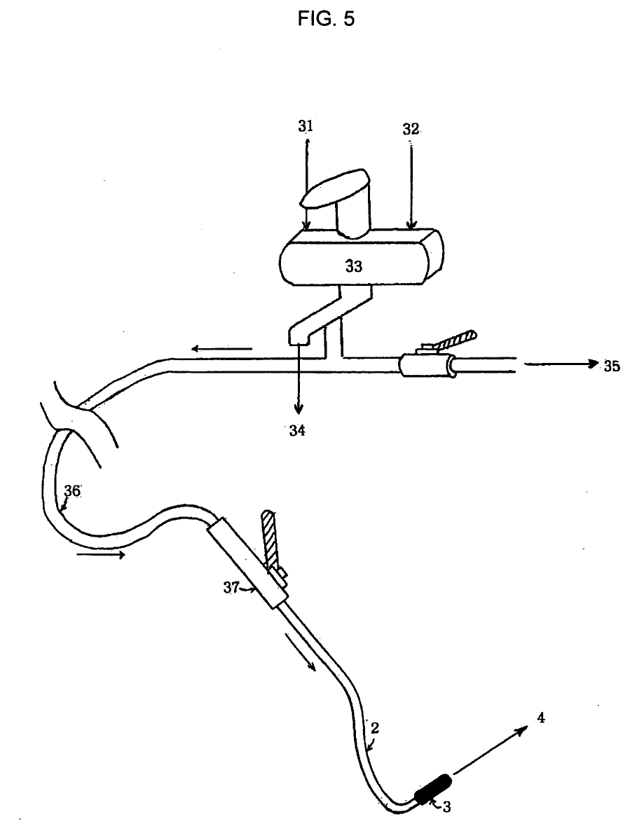

As shown in FIGS. 4A, 4B and 5, a vaginal irrigator is composed of an elastic bottle (1) in the shape of a cylinder or a truncated cone or a combination of both, a neck of spray nozzle (2) of about 100-400 mm long which is removably connected to the bottle cap, and a spray nozzle (3) of about 10-50 mm long. FIG. 4A shows a vaginal irrigator in use with the spray nozzle being inserted into the vagina, while FIG. 4B shows its position for anus cleansing. The neck of spray nozzle is bended sharply 60-120 degrees following the shape of the vaginal inlet after maintaining straight for 10-60 mm. Then, once again it is bended smoothly 10-90 degrees to the same direction following the shape of abdomen. Then, it is bended smoothly 0-60 degrees to the opposite direction in order to make the operation of the irrigator convenient. The neck of spray nozzle is made of strong but pliable materials such as aluminum so that the bending angles can be finely adjusted to fit each individual's sitting posture on the toilet seat. As shown in FIGS. 4A and 4B, the spray nozzle and the neck of spray nozzle together look like a cobra whose head is raised when the vaginal irrigator is turned 135 degrees counter-clockwise. Holding the elastic bottle as a handle one can easily find the inlet of the vagina by the touch of the spray nozzle and insert the spray nozzle into the vagina. With the spray nozzle in the vagina, one can easily wash out the vagina thoroughly moving the spray nozzle front and back. Due to the neck of spray nozzle (2), which is bended for 60-120 degrees sharply following the shape of the vaginal inlet, the spray nozzle (3) is prevented from being inserted too deeply into the vagina. Differing from existing portable irrigators, no air is sprayed before all the washing liquid is finished. This is because the elastic bottle is positioned upside down and the washing liquid gathers at the outlet of the bottle. Instead of an elastic bottle, the neck of the spray nozzle can be connected to water pipes in the bathroom with a flexible hose and a pressure control valve as shown in FIG. 5. As shown in FIG. 4B, these vaginal irrigators can be used to cleanse the anus as well.

Vaginal Scrubbing Sponge

As shown in FIG. 6, the sponge (1) is made of soft, elastic, absorptive and adsorptive material. It has the shape of sphere, cylinder, elliptical sphere or the likes whose diameter is about 10-30 mm and whose length is about 10-70 mm. The sponge is partially perforated through its axis. Once the handle is inserted into the hole a wide elastic ring or a single-sided adhesive tape is wound around compressing partially the sponge on the edge. The total length of the sponge and the handle is 70-200 mm. The handle is made thinner where it is wound with the wide elastic ring or a single-sided adhesive tape so that the thickness becomes even when the ring or the tape is wound. Furthermore, surface-coating materials such as vanish or adhesive are applied to fill the gap between the connecting parts to make the connection smoother in order to avoid scratches on the vaginal surface. The end of the handle which is buried in the sponge has the shape of sphere or the like whose diameter is larger than that of the handle. This is not only to avoid accidental scratches by the bare end of the handle when the sponge is pulled off but also to make the scrubbing action easier. Even though it is recommended to use the vaginal scrubbing sponge along with the vaginal irrigator, one can still accomplish sufficient vaginal cleansing with the vaginal scrubbing sponge alone by repeated scrubbing and absorbing in the vagina followed by washing and wringing the sponge outside of vagina for several times.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

EXPERIMENT 1: The fact that distilled water is an excellent spermicide was proven experimentally as follows: (1) One drop of semen was spread on a slide glass with a cotton swab and active movements of sperms were confirmed. (2) Upon mixing with a drop of distilled water, 100% of sperms stood still immediately. (3) The sperms failed to show any activity upon addition of saline solution. All sperms might be dead by explosive expansion of the sperm cell. While not wishing to be bound by any particular theory, the inventor believes that this is due to a lot of water smeared into the cell due to the difference in osmotic pressure between the cell and distilled water.

EXPERIMENT 2: The fact that saline solution does not affect the sperm's activity was verified experimentally as follows: (1) One drop of semen was spread on a slide glass with a cotton swab and active movements of sperms were confirmed. (2) Upon mixing with a drop of saline solution, no change in the activity of sperms was observed.

EXPERIMENT 3: The fact that water-soluble polymers do not kill sperms, but immobilize them by their high viscosity was proven experimentally as follows: (1) One drop of semen was spread on a slide glass with a cotton swab and active movements of sperms were confirmed. (2) Upon mixing with a drop of water-soluble polymer solution, all sperms were immobilized and did not move forward except that some of them could only shake their heads. (3) Upon decreasing the viscosity by adding several drops of saline solution, the sperms regained their original activity.

EXPERIMENT 4: The fact that polymer solutions made from distilled water and water-soluble polymers exterminated sperms was verified experimentally as follows: (1) One drop of semen was spread on a slide glass with a cotton swab and active movements of sperms were confirmed. (2) Upon mixing with a drop of water-soluble polymer solution, all sperms came to a standstill without any movement in their heads or tails. (3) Even after reducing the viscosity by adding several drops of saline solution, the sperms failed to regain their original activities.

EXPERIMENT 5: The fact that ordinary tap water, in which the salt content is about 9 ppm is also an excellent spermicide was proven experimentally as follows: (1) One drop of semen was spread on a slide glass with a cotton swab and active movements of the sperms were confirmed. (2) Upon mixing with several drops of tap water, 100% of the sperms stood still immediately. (3) The sperms failed to show any activity upon addition of saline solution.

In view of the above five experiments, while not wishing to be bound to any particular theory, the inventor believes that it can be concluded as follows: (a) In contrast with other spermicides, distilled water kills sperms not by chemical but by physical phenomenon. (b) It is necessary to enhance the viscosity of distilled water for practical uses of distilled water as a spermicide, in which water soluble polymers such as cellulose derivatives can be used without appreciable changes in osmotic pressure of the distilled water. (c) Water-soluble polymer solutions immobilize sperms by their high viscosities. (d) Cervices can be protected from sperms' invasion if water-soluble polymer solutions of high viscosity are injected deep into vaginas. (e) Water-soluble polymer solutions injected into vaginas can be easily washed out with the vaginal irrigator and the vaginal scrubbing sponge of the present invention. (f) Water-soluble polymer solutions are highly lubricous and non-irritant to the vagina and the male urethra.

In order to raise the viscosity of distilled water, the present inventor employed water-soluble polymers such as polyvinyl alcohol (PVA) and cellulose derivatives (e.g., sodium carboxymethyl cellulose (SCMC), hydroxyethyl cellulose (HEC), and hydroxypropyl methyl cellulose (HPMC)). The solution viscosity depends upon concentrations and molecular weights of the polymers. In most of the cases, less than 20 wt % of these water-soluble polymers gave viscosities high enough to be injected into male urethras and vaginas.

### Example 2

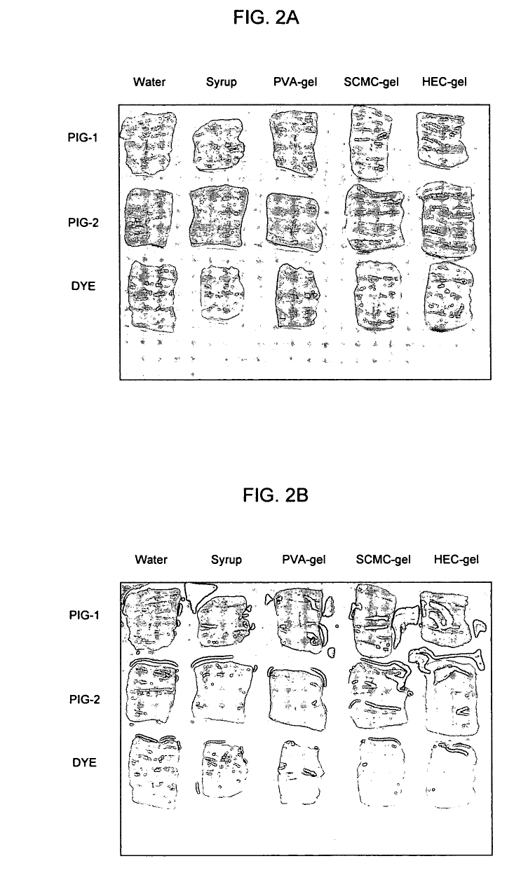

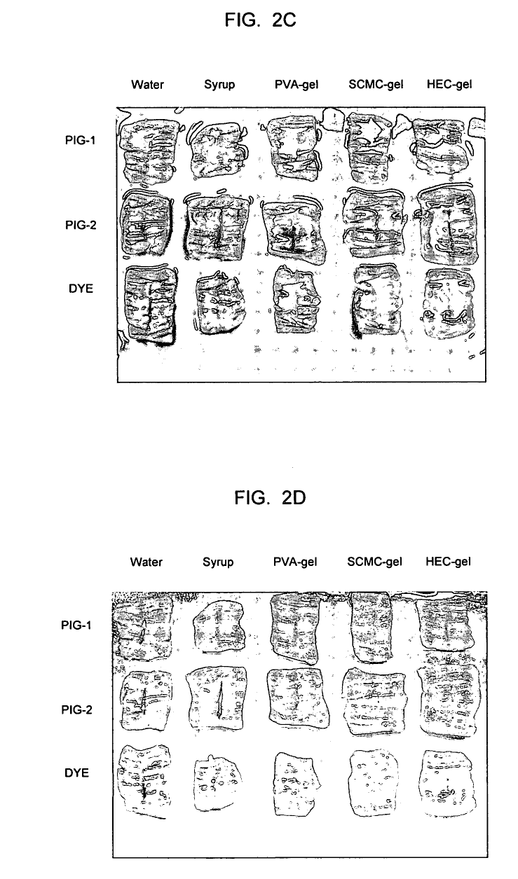

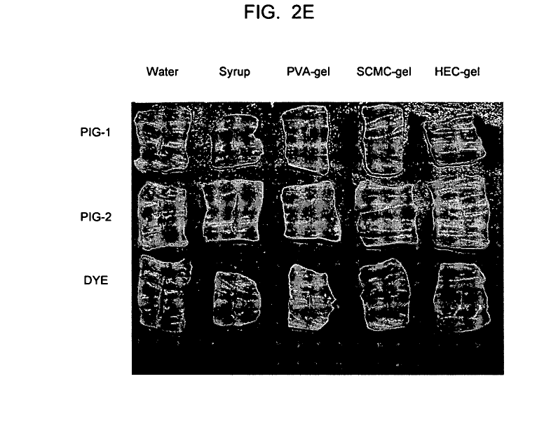

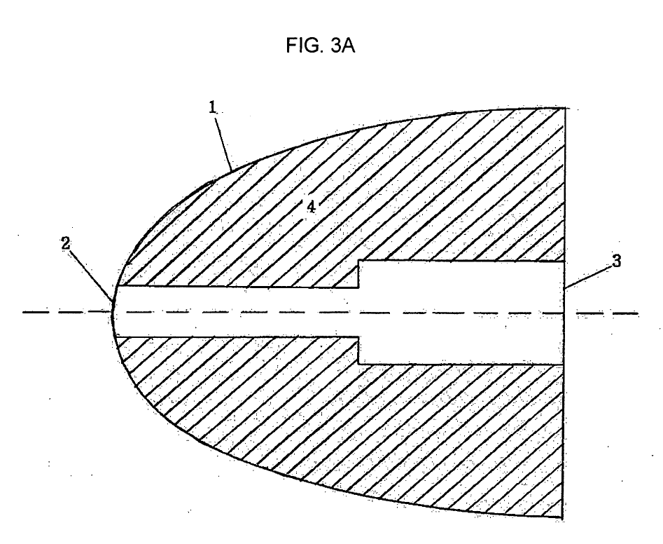

EXPERIMENT 6: Two pigments whose particle size distributions correspond to sizes of STD-causing germs including HIV (diameter=125 nm) were chosen. The blue pigment (PIG-1) had the particle size distribution of 100-20,000 nm, while the violet pigment (PIG-2) had the particle size distribution of 30-500 nm. For the purpose of comparison, a blue dye ink (DYE) (molecular diameter=2.7 nm) was used as well. Instead of the human vagina, the swine vagina was used. FIG. 2a shows the 15 vaginal segments of swine, in which each segment was incised once with a razor blade in order to simulate an accidental injury which might occurs during coitus. Then, the vaginal segments in the left column were wetted with water, while those in the second, the third, the fourth and the fifth column were applied with corn syrup (SYRUP, 55% maltose), a composition made from polyvinyl alcohol (PVA-gel), a composition made from sodium carboxymethyl cellulose (SCMC-gel) and a composition made from hydroxyethyl cellulose (HEC-gel), respectively, as shown in FIG. 2B. After that, the blue pigment (PIG-1) was applied on the vaginal segments in the upper row, while the violet pigment (PIG-2) and the blue dye ink (DYE) were applied on the vaginal segments in the middle and the lower row, respectively, as shown in FIG. 2C. The pigments and the dye ink applied on the vaginal segments were rubbed 50 times violently with the index finger being pressed in an attempt to simulate the frictional movement of coitus. Then, these vaginal segments were rinsed out only with running water as shown in FIG. 2D. Thereafter that, they were scrubbed off and cleansed out with the vaginal scrubbing sponge of the present invention as shown in FIG. 6. The result is shown in FIG. 2E.

By comparing FIG. 2A with FIG. 2D and FIG. 2E, the present inventor discovered the following facts: (a) Pigments applied on PVA-gel, SCMC-gel and HEC-gel were completely rinsed out even before being scrubbed off both from the intact vaginal surfaces and incisions. This fact indicates that those pigment particles (30-20,000 nm) did not penetrate the 3-dimensional network (interval=4-15 nm) formed with water-soluble polymers as shown in FIG. 1 in order to arrive and attach onto the vaginal surfaces in spite of violent turbulence made by the index finger. Therefore, water soluble polymers provide physical blocking walls on vaginal surfaces against microbial particles and they can be used to prevent STDs including AIDS in despite of injuries. (b). Pigments applied on both low-viscosity water and high-viscosity corn syrup still remained after rinsing and scrubbing. In particular, pigments on incisions remained heavily. This is an experimental proof that epidermal injuries enable microbial invasions easy. Therefore, it can be said that low molecular weight substances such as water, corn syrup or glycerin do not form effective physical blocking walls against microbes regardless of their viscosity. (c) The dye applied on water and corn syrup remained heavily after being rinsed and scrubbed on both intact vaginal surfaces and incisions. (d) The dye applied on PVA-gel, SCMC-gel and HEC-gel remained slightly after being rinsed and scrubbed on both intact vaginal surfaces and incisions. This fact indicates that some of the dye molecules (molecular diameter=2.7 nm) penetrate the 3-dimensional network (interval=4-15 nm) of water-soluble polymers as shown in FIG. 1 and arrive to the vaginal surfaces to be attached.

### Example 3

CLINICAL TEST 1: A middle-aged bachelor tested Urethral Condoms about one hundred and seventy times with about eighty females whose average chance of STD infection was reported to be 36.4%. By injecting 2 ml of the compositions (without colloidal silver) of the present invention only into his urethra before each coitus, he had not been infected with an STD even a single time.

CLINICAL TEST 2: A 25-year-old single woman with an irregular menstrual cycle and an experience in artificial abortion had been successful in contraception for 16 months by injecting 12 ml of the compositions (without colloidal silver) of the present invention into her vagina before each intercourse and then by washing out with the vaginal irrigator of the present invention.

### Example 4

### Manufacture of Compositions for Prevention of Pregnancy and STDs

First, 500 ml of distilled water is made to be colloidal silver whose particle size and concentration are 2-15 nm and 6 ppm respectively by an electrolysis apparatus with silver electrodes of purity more than 99.99%. Then, 250 ml of it is poured into in a wide-mouth bottle. In order to make its concentration to be 2.59 wt. %, 6.65 g of water-soluble polymer (Hercules Chemicals Co.'s SCMC, Grade FVH-9A) in the form of powder is added to this while being shaken frequently at the room temperature. After 48 hours with frequent shakings, it turns into a viscous and slippery gel whose Brookfield Viscosity is 83,000 mPa*s. For the stability of colloidal silver, all experimental utensils must be made of either glass or plastics.

Compositions without colloidal silver can be manufactured when 0.5-10.0 g of cellulose derivatives such as SCMC, HEC, and HPMC in the form of powder are added slowly to 100 ml of distilled water while being shaken. Then, they are left for 5-72 hours with frequent shakings at the room temperature. Compositions without colloidal silver can also be manufactured when 7.0-20.0 g of PVA in the form of powder is added slowly to 100 ml of distilled water and heated up to about 80 degrees centigrade for about 30 minutes with continuous stirring. These compositions manufactured as the above can perform their roles as prophylactics for conception and STDs alone or as a mixture of them.

### Example 5

### Liquid Injector into the Male Urethra

It is composed of a pressure generator and an injection nozzle. The injection nozzle has the shape of or similar to one half of the ellipsoid whose short, middle, and long radii are 6.5 mm, 6.5 mm, and 15 mm respectively. A syringe or a collapsible tube similar to the toothpaste tube can be the pressure generator.

### Example 6

### Vaginal Irrigators

It is composed of an elastic bottle in the shape of a combination of a cylinder and a truncated cone, a neck of spray nozzle made of aluminum which is 190 mm long and is attached to the cap of the elastic bottle, and a spray nozzle of 30 mm long. They are connected removably in the above-mentioned order. The neck of spray nozzle maintains straight for 20 mm, then sharply bends 90 degrees followed by another smooth bend of 25 degrees, again followed by the second smooth bend of 25 degrees to the opposite direction. Instead of an elastic bottle, the neck of the spray nozzle can be connected to a water pipe with a flexible hose and a pressure control valve.

### Example 7

### Vaginal Scrubbing Sponge

A cylindrical sponge (diameter=23 mm, height=35 mm) is partially perforated through its axis. Once the handle (diameter=7 mm, length=150 mm) is inserted into the hole, a single-sided adhesive tape is wound around compressing partially the sponge on the edge. The handle is made 1 mm thinner where the single-sided adhesive tape is wound so that the thickness becomes even when the tape is wound. Then, a cyanoacrylate instant adhesive is applied filling the gap formed between the edge of the adhesive tape and the handle to make the connection smoother. The end of the handle which is buried in the sponge has the shape of sphere whose diameter is 12 mm.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A composition, comprising:
a quantity of distilled water; and
a water-soluble polymer, wherein the concentration of the water-soluble polymer is about 0.5% to about 20.0% by weight.

2. The composition of claim 1, wherein the water-soluble polymer is selected from the group consisting of polyvinyl alcohol (PVA), sodium carboxymethyl cellulose (SCMC), hydroxyethyl cellulose (HEC), hydroxypropyl methyl cellulose (HPMC) and combinations thereof.

3. The composition of claim 1, further comprising a quantity of colloidal silver to bring the concentration of the colloidal silver to about 1 to about 10 ppm by weight.

4. A method of using the composition of claim 1 to prevent or reduce the likelihood of pregnancy and/or to prevent or reduce the likelihood of contracting a sexually transmitted disease, comprising:
providing the composition of claim 1; and
applying the composition onto or into to a male urethra or into a vagina prior to sexual intercourse.

5. A device for injecting a composition into the male urethra, comprising:
an injection nozzle adapted for insertion into a male urethra; and
a pressure generator,
wherein the diameter of the distal end of the injection nozzle is smaller than the diameter of the male urethral outlet, the diameter of the proximal end of the injection nozzle is larger than the diameter of the male urethral outlet and the diameter of the injection nozzle increases from the distal end to the proximal end whereby a gap between the urethral outlet and the injection nozzle is sealed when the distal end of the injection nozzle is partially inserted into the urethra and pressed against the urethral outlet.

6. The device of claim 5, wherein the shape of the injection nozzle is substantially similar to the shape of one-half of an ellipsoid.

7. The device of claim 5, wherein the ellipsoid is defined by a first radius that is about 4 to about 9 mm, a second radius that is about 5 to about 12 mm and a third radius that is about 10 to about 30 mm.

8. A method of using the device of claim 5 to prevent or reduce the likelihood of pregnancy and/or to prevent or reduce the likelihood of contracting a sexually transmitted disease, comprising;
providing the device of claim 5 and a composition comprising distilled water and a water-soluble polymer;
placing the composition into device; and
using the device to inject the composition into the male urethra.

9. A vaginal irrigation device, comprising:
a spray nozzle;
a neck of the spray nozzle; and
optionally, an elastic bottle,
wherein the neck of the spray nozzle is removably connected to the spray nozzle and optionally, removably connected to the elastic bottle, and
wherein the shape of the neck of the spray nozzle comprises a straight segment of about 10 mm to about 60 mm measured from the distal end, followed by a sharp curve of about 60 to about 120 degrees, followed by a first gradual curve of about 10 to about 90 degrees in the direction of the distal end, followed by a second gradual curve of about 0 to about 60 degrees in the opposite direction.

10. The vaginal irrigation device of claim 9, wherein the length of the spray nozzle is about 10 to about 50 mm and the length of the neck of the spray nozzle is about 100 to about 400 mm.

11. The vaginal irrigation device of claim 9, wherein the neck of the spray nozzle is further connected to a water pipe with a flexible hose comprising a pressure control valve.

12. The vaginal irrigation device of claim 9, wherein the neck of the spray nozzle comprises a strong and pliable material.

13. A method of using the vaginal irrigation device of claim 9, comprising:
providing the vaginal irrigation device of claim 9, and
using the vaginal irrigation device to cleanse the vagina.

14. The method of claim 13, further comprising using the vaginal irrigation device to remove a composition comprising distilled water and a water-soluble polymer from the vagina.

15. A vaginal scrubbing sponge device, comprising:
a sponge;
a handle; and
a wide elastic ring or a single-sided adhesive tape,
wherein the sponge is connected to the distal end of the handle with the wide elastic ring or the single-sided adhesive tape whereby the sponge is partially compressed on the proximal edge.

16. The vaginal scrubbing sponge device of claim 15, wherein the diameter of the portion of the handle wound with the wide elastic ring or the single-sided adhesive tape is smaller than the diameter of an adjacent portion of the handle.

17. The vaginal scrubbing sponge device of claim 15, further comprising a surface-coating material capable of filling a gap formed between the edge of the wide elastic ring or the single-sided adhesive tape and the handle to make a smooth connection between the sponge and the handle.

18. The vaginal scrubbing sponge device of claim 15, wherein the shape of the distal end of the handle is spherically or substantially spherical, or wherein the diameter of the distal end of the handle is larger than the diameter of a portion of the handle that is adjacent to the distal end.

19. A method of using the vaginal scrubbing sponge device of claim 15, comprising:
providing the vaginal scrubbing sponge device of claim 15; and
using the vaginal scrubbing sponge device to cleanse the vagina.

20. The method of claim 19, further comprising using the vaginal scrubbing sponge device to remove a composition comprising distilled water and a water-soluble polymer from the vagina.
